(19) United States
(12) Patent Application Publication
(10) Pub. No.: US 2020/0016076 A1
(43) Pub. Date: Jan. 16, 2020

(54) COMPOSITION FOR THE TREATMENT OF DUCHENNE MUSCULAR DYSTROPHY

(71) Applicant: Summit (Oxford) Limited, Abingdon Oxfordshire (GB)

(72) Inventor: Graeme Horne, Summit (Oxford) Limited (GB)

(21) Appl. No.: 16/144,809

(22) Filed: Sep. 27, 2018

Related U.S. Application Data
(63) Continuation of application No. PCT/GB2017/050884, filed on Mar. 29, 2017.

(30) Foreign Application Priority Data
Mar. 30, 2016 (GB) 1605383.7
Jul. 26, 2016 (GB) 1612920.7

(51) Int. Cl. A61K 9/10; A61K 31/423; A61K 47/38; A61K 9/16; A61P 21/00; A61K 9/19

(52) U.S. Cl. CPC A61K 9/10; A61K 31/423; A61K 9/19; A61K 9/1682; A61P 21/00; A61K 47/38

(57) ABSTRACT

Disclosed are amorphous solid dispersions (ASDs) comprising the compound 5-(ethylsulfonyl)-2-(naphthalen-2-yl)benzo[d]oxazole (ezutromid) and a polymer. The ASDs find application in the treatment or prophylaxis of Duchenne muscular dystrophy and Becker muscular dystrophy.

### TECHNICAL FIELD

This invention relates to amorphous solid particle compositions comprising 5-(ethylsulfonyl)-2-(naphthalen-2-YL)benzo[d]oxazole (SMT C1100, now also designated by the international nonproprietary name ezutromid), to processes for preparing the compositions, and to various therapeutic uses of the compositions. Also provided is a method of treatment of Duchenne muscular dystrophy or Becker muscular dystrophy using the compositions.

### BACKGROUND OF THE INVENTION

Duchenne muscular dystrophy (DMD) is a common, genetic neuromuscular disease associated with the progressive deterioration of muscle function, first described over 150 years ago by the French neurologist, Duchenne de Boulogne, after whom the disease is named. DMD has been characterized as an X-linked recessive disorder that affects 1 in 3,500 males caused by mutations in the dystrophin gene. The gene is the largest in the human genome, encompassing 2.6 million base pairs of DNA and containing 79 exons, Approximately 60% of dystrophin mutations are large insertion or deletions that lead to frameshift errors downstream, whereas approximately 40% are point mutations or small frameshift rearrangements. The vast majority of DMD patients lack the dystrophin protein. Becker muscular dystrophy is a much milder form of DMD caused by reduction in the amount, or alteration in the size, of the dystrophin protein. The high incidence of DMD (1 in 10,000 sperm or eggs) means that genetic screening will never eliminate the disease, so an effective therapy is highly desirable.

Upregulation of utrophin, an autosomal paralogue of dystrophin, has been proposed as a potential therapy for DMD (Perkins & Davies, Neuromuscul Disord, S1: S78-S89 (2002), Khurana & Davies, Nat Rev Drug Discov 2:379-390 (2003)). When utrophin is overexpressed in transgenic mdx mice it localizes to the sarcolemma of muscle cells and restores the components of the dystrophin-associated protein complex (DAPC), which prevents the dystrophic development and in turn leads to functional improvement of skeletal muscle. Adenoviral delivery of utrophin in the dog has been shown to prevent pathology. Commencement of increased utrophin expression shortly after birth in the mouse model can be effective and no toxicity is observed when utrophin is ubiquitously expressed, which is promising for the translation of this therapy to humans. Upregulation of endogenous utrophin to sufficient levels to decrease pathology might be achieved by the delivery of small diffusible compounds.

Ezutromid is a small molecule utrophin upregulator that has the potential to be a universal treatment for DMD.

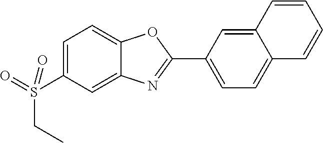 [SMILES: CCS(=O)(=O)c1ccc2oc(-c3ccc4ccccc4c3)nc2c1]

### 5-(ethylsulfonyl)-2-(naphthalen-2-yl)benzo[d]oxazole (ezutromid)

The synthesis and therapeutic use of this compound is described in our earner WO2007/091106, while its various polymorphic forms and processes for the production of such forms are described in WO2009/021748.

The compound acts in synergy with corticosteroids, including prednisone, prednisolone and deflazacort, to reduce exercise-induced fatigue in mouse models of DMD (see our earlier WO2009/019504).

It is desirable to improve the bioavailability of ezutromid and there is a need for oral pharmaceutical formulations which improve drug delivery. A liquid pharmaceutical composition which permits improved ezutromid uptake which comprises an aqueous suspension of nanoparticulate ezutromid is described in our earlier WO/2013/167737.

It has now been surprisingly discovered that the oral bioavailability of ezutromid may be even further improved by using amorphous solid dispersions (ASDs). Bioavailability enhancement may be achieved by improving the dissolution kinetics of ezutromid and/or by increasing the maximum concentration of ezutrornid in solution.

ASDs are reviewed in Lee et al (2014), Current Pharmaceutical Design 20: 303-324 (the content of which is incorporated herein by reference).

### BRIEF DESCRIPTION OF THE DRAWINGS

FIG. 1 shows the XRPD pattern for Form I of the drug substance.

FIG. 2 shows the DSC trace.

FIG. 3 shows results from the TGA of Form I.

FIG. 4 shows a chemical synthesis.

FIG. 5 shows the XRPD profile of Form II.

FIG. 6 shows the FT-IR profile.

FIG. 7 shows the Raman spectrum of ezutromid.

FIG. 8 shows proportionality in exposures between DMD boys and healthy volunteers.

### SUMMARY OF THE INVENTION

According to the invention there is provided an amorphous solid dispersion (ASD) comprising the compound 5-(ethylsulfonyl)-2-(naphthalen-2-yl)benzo[d]oxazole (SMT C1100, ezutromid) and a polymer.

Ezutromid for use in the compositions may be synthesised by any suitable methods, including those described herein and in WO2007/091106, WO2009/021748 and WO2009/019504.

The amorphous solid dispersions of the invention comprise dispersed ezutromid in an amorphous form. Preferably, ezutromid is dispersed uniformly throughout the polymer. The ezutromid may be present in a substantially non-crystalline state: for example, the ASDs of the invention may be solid solutions. Preferably, less than 20%, 15%, 10%, 5%, 1% or 0.1% by weight of the ezutroniid in the ASD is in a crystalline form.

The polymer may be in the form of a polymer matrix in which amorphous ezutromid is dispersed.

The polymer may be a water soluble polymer. In certain embodiments, the polymer is a solubilizing polymer. The polymer may inhibit amorphous ezutromid recrystallization in the solid-state and/or promote supersaturation in the solution state upon dissolution.

Any suitable polymer may be employed, but preferred may be polymers which comprise, or consist essentially of, a cellulosic or non-cellulosic polymer.

Thus, in certain embodiments the polymer comprises, or consists essentially of, a cellulosic polymer, optionally selected from the group consisting of ionizable cellulosic polymers, non-ionizable cellulosic polymers, neutralized acidic cellulosic polymers and blends thereof.

For example, the polymer may comprise, or consist essentially of, a non-cellulosic polymer, optionally selected from the group consisting ionizable non-cellulosic polymers, non-ionizable ionizable non-cellulosic polymers, neutralized acidic non-cellulosic polymers and blends thereof.

In certain embodiments, the polymer is a chemically modified cellulose and/or cellulose ether.

Not limiting examples of suitable polymers for use according to the invention therefore include, without limitation, chemically modified cellulose and/or cellulose ethers selected from: alkylcellulose (for example methylcellulose, ethylcellulose and propylcellulose); hydroxyalkylcellulose (for example hydroxymethylcellulose, hydroxyethylcellulose and hydroxypropylcellulose); hydroxyalkylalkylcellulose (for example hydroxyethylmethylcellulose (HEMC) and hydroxypropylmethylcellulose (HPMC)); carboxyalkylcellulose (for example carboxymethylcellulose (CMC), carboxymethylethylcellulose, carboxymethyl hydroxyethylcellulose (CMH EC), hydroxyethylcarboxymethylcellulose (HEC C) and sodium carboxymethylcellulose); cellulose acetate phthalate (CAP); cellulose acetate trimellitate, hydroxypropylmethylcellulose acetate (HPMCA); hydroxypropylmethylcellulose phthalate (HPMCP); hydroxypropylmethylcellulose acetate succinate (HPMCAS), polyvinyl alcohols having repeat units in hydrolyzed form, polyvinyl pyrrolidone, poloxamers, polyvinylpyrrolidone, polyethylene glycol, polyethylene glycol based copolymer, polyacrylic acids and salts thereof, polyvinylalcohol, polyacrylamides copolymer, methacrylic acid copolymer, methacrylate copolymer, pectines, chitin and chitosan derivatives, polyphosphates, polyoxazoline, polysaccharides and mixtures thereof.

The polymer may comprise, or consist essentially of, HPMCAS. In such embodiments, the HPMCAS may be selected from subtypes L, M and H, for example subtype M. In certain embodiments, the HPMCAS comprises a combination of two or more subtypes selected from subtypes L, M and H. The HPMCAS may have a succinate/acetate ratio (CAR.) which is selected to optimize supersaturation of ezutromid in the solution state upon dissolution.

Also contemplated for use according to the invention are HPMCAS analogues, for example as described in WO 2014/182710 and Ting et al. (2015) ACS Biomater. Sci. Eng. 1: 978-990 (the content of which is incorporated herein by reference).

Suitable HPMCAS analogues therefore include acrylate polymers comprising at least two monomeric units, wherein the first monomeric unit is derived from the monomers selected from (a), (b), (c) and (d):

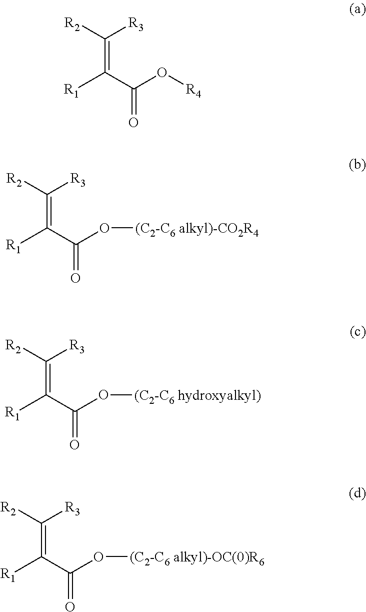

and the second monomeric unit is derived from a monomer of the formula:

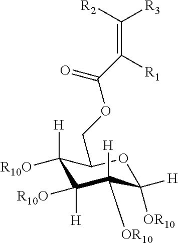

wherein
R₁, R₂ and R₃ are independently H or methyl;
R₄ is H or C₁-C₆ alkyl;
R is C₁-C₆ alkyl; and
at each occurrence, R₁₀ is independently H, C₁-C₄ alkyl, C₂-C₄ alkanoyl, C₂-C₅
alkenoyl, —C₁-C₄ alkyl-aryl, or -alkanoylaryl;
and wherein the C₂-C₆ hydroxyalkyl group has one or two OH groups,

In all embodiments of the invention, the polymer may comprise a blend of different polymers.

The dispersion of the invention may take the form of solid polymeric particles (SPPs), wherein the polymer forms a matrix containing dispersed ezutromid. In such embodiments, the SPPs may have a D₅₀ particle size less than 20 μm; a D₉₀ particle size less than: 40 μm; or a D₅₀ particle size less than 20 μm and a 0₉₀ particle size less than 40 μm. Alternatively; or in addition, the SPPs may have the particle size distribution (PSD): D₁₀<10 μm, D₅₀<20 μm and D₉₀<40 μm.

The ezutromid may be present at a concentration of: at least 10% wt/wt; at least 20% wt/wt; at least 30% wt/wt; at least 40% wt/wt; at least 50% wt/wt; or about 50% wt/wt.

In preferred embodiments, the ezutromid is stable in the amorphous state upon storage, for example for at least 1 week, 2 weeks, 4, weeks, 1 month, 3 months, 6 months or 1 year at room temperature. In such embodiments, it is preferred that less than 20%, 15%, 10%, 5%, 1% or 0.1% by weight of the amorphous, non-crystalline ezutromid present in the ASD recrystallizes upon storage, for example during storage at room temperature for at least 1 week, 2 weeks, 4, weeks, 1 month, 3 months, 6 months or 1 year.

Any suitable method may be used to prepare the dispersion of the invention, including spray drying, freeze drying, hot melt extrusion or co-precipitation.

In another aspect, the invention contemplates a pharmaceutical composition comprising the dispersion of the invention and a pharmaceutically acceptable excipient.

In another aspect, the invention contemplates a dosage form comprising the dispersion or pharmaceutical composition of the invention.

The dosage form may take the form of a tablet or granules. In such embodiments, the dosage form may further comprise an intragranular and/or extragranular excipient. Such excipients may be selected from: fillers, disintegrants, lubricants, glidants, surfactants and mixtures thereof. Examples include microcrystalline cellulose, croscarmellose sodium, sodium lauryl sulfate, sodium stearyl fumarate and mixtures thereof.

The dosage forms of the invention may comprise the dispersion of the invention suspended in an aqueous vehicle. In such embodiments, the aqueous vehicle may comprise a fat. Suitable aqueous vehicles therefore comprise milk, for example full fat or semi-skimmed milk.

The dosage form of the invention is preferably adapted for oral administration.

In another aspect the invention contemplates a process for producing a dispersion, pharmaceutical composition or dosage form of the invention comprising: (a) spray drying; (b) freeze drying; (c) hot melt extrusion, or (d) co-precipitation, of said ezutromid and polymer.

The process may comprise the steps of: (a) dissolving the polymer and ezutromid in a solvent system to form a feed stock solution; and (b) spray drying the feed stock solution to form SPPs containing ezutromid dispersed therein. The solvent system may comprise acetone. The process may further comprise the step (c) of collecting the SPPs, for example by means of a cyclone, electrostatic precipitator or bag filter. The process may also further comprise the step of compacting or tableting the SPPs.

Also contemplated is a foodstuff comprising a dispersion, pharmaceutical composition or dosage form of the invention.

Also contemplated are compositions produced, obtained, or obtainable by, the processes of the invention.

In another aspect, the invention contemplates a dispersion, pharmaceutical composition, dosage form, foodstuff or composition as defined above for use in therapy or prophylaxis.

In another aspect, the invention contemplates a dispersion, pharmaceutical composition, dosage form, foodstuff or composition as defined above for use in the treatment or prophylaxis of Duchenne muscular dystrophy or Becker muscular dystrophy.

In another aspect, the invention contemplates the use of a dispersion, pharmaceutical composition, dosage form, foodstuff or composition as defined above for the manufacture of a medicament for use in the treatment or prophylaxis of Duchenne muscular dystrophy or Becker muscular dystrophy.

In another aspect, the invention contemplates a method for the treatment or prophylaxis of Duchenne muscular dystrophy or Becker muscular dystrophy in a patient in need thereof, comprising orally administering to the patient an effective amount of a dispersion, pharmaceutical composition, dosage form, foodstuff or composition as defined above.

Other aspects of the invention are defined in the claims attached hereto.

### DETAILED DESCRIPTION OF THE INVENTION

All publications, patents, patent applications and other references mentioned herein are hereby incorporated by reference in their entireties for all purposes as if each individual publication, patent or patent application were specifically and individually indicated to be incorporated by reference and the content thereof recited in full.

Definitions and General Preferences

Where used herein and unless specifically indicated otherwise, the following terms are intended to have the following meanings in addition to any broader (or narrower) meanings the terms might enjoy in the art:

Unless otherwise required by context, the use herein of the singular is to be read to include the plural and vice versa. The term “a” or “an” used in relation to an entity is to be read to refer to one or more of that entity. As such, the terms “a” (or “an”), “one or more,” and “at least one” are used interchangeably herein.

As used herein, the term “comprise,” or variations thereof such as “comprises” or “comprising,” are to be read to indicate the inclusion of any recited integer (e.g. a feature, element, characteristic, property, method/process step or limitation) or group of integers (e.g. features, element, characteristics, properties, method/process steps or limitations) but not the exclusion of any other integer or group of integers. Thus, as used herein the term “comprising” is inclusive or open-ended and does not exclude additional, unrecited integers or method/process steps.

The phrase “consisting essentially of” is used herein to require the specified integer(s) or steps as well as those which do not materially affect the character or function of the claimed invention.

As used herein, the term “consisting” is used to indicate the presence of the recited integer (e.g. a feature, element, characteristic, property, method/process step or limitation) or group of integers (e.g. features, element, characteristics, properties, method/process steps or limitations) alone.

As used herein, the term “treatment” or “treating” refers to an intervention (e.g. the administration of an agent to a subject) which cures, ameliorates or lessens the symptoms of a disease or removes (or lessens the impact of) its cause(s). In this case, the term is used synonymously with the term “therapy”.

Additionally, the terms “treatment” or “treating” refers to an intervention (e.g. the administration of an agent to a subject) which prevents or delays the onset or progression of a disease or reduces (or eradicates) its incidence within a treated population. In this case, the term treatment is used synonymously with the term “prophylaxis”.

The term “subject” (which is to be read to include “individual”, “animal”, “patient” or “mammal” where context permits) defines any subject, particularly a mammalian subject, for whom treatment is indicated. Mammalian subjects include, but are not limited to, humans.

As used herein, an effective amount or a therapeutically effective amount of a compound defines an amount that can be administered to a subject without excessive toxicity, irritation, allergic response, or other problem or complication, commensurate with a reasonable benefit/risk ratio, but one that is sufficient to provide the desired effect, e.g. the treatment or prophylaxis manifested by a permanent or temporary improvement in the subject's condition. The amount will vary from subject to subject, depending on the age and general condition of the individual, mode of administration and other factors. Thus, while it is not possible to specify an exact effective amount, those skilled in the art will be able to determine an appropriate “effective” amount in any individual case using routine experimentation and background general knowledge. A therapeutic result in this context includes eradication or lessening of symptoms, reduced pain or discomfort, prolonged survival, improved mobility and other markers of clinical improvement. A therapeutic result need not be a complete cure.

As used herein, a “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

A “pharmaceutical composition” is a composition in a form, concentration and level of purity suitable for administration to a patient (e.g. a human or animal patient) upon which administration it can elicit the desired physiological changes. Pharmaceutical compositions are typically sterile and/or non-pyrogenic. The term non-pyrogenic as applied to the pharmaceutical compositions of the invention defines compositions which do not elicit undesirable inflammatory responses when administered to a patient.

The particle sizes referenced herein may be measured by any conventional particle size measuring technique known to those skilled in the art. Such techniques include, for example, sedimentation field flow fractionation, photon correlation spectroscopy, light scattering (e.g. laser diffraction) and disk centrifugation.

As used herein, the term “solubilizing polymer” defines a polymer which is capable of: (a) improving the dissolution kinetics of ezutromid and/or (b) increasing the maximum concentration of ezutromid in solution when associated with ezutromid in the form of an ASD.

Methods for Making the ASDs of the Invention

ASDs can be prepared using a variety of manufacturing techniques. Hot melt extrusion and spray drying are convenient techniques for manufacturing large quantities, while lyophilization (freeze drying) or supercritical fluid processing may also be suitable.

Hot Melt Extrusion

Hot melt extrusion is now widely used for manufacturing ASDs. Both ram and screw extrusion, may be employed. In both cases, the ezutromid and polymer are added to a heated vessel, softened and forced through a die using a piston. Depending on the size of the die and the application, the extrudates can be processed by appropriate techniques into different dosage forms.

Spray Drying

Spray drying involves atomization, drying and collection of the powder. During atomization, a fine mist with a large surface area is sprayed into a heated chamber. The formation of fine droplets helps to promote heat transfer and immediate evaporation of the liquid phase.

Formulation

Pharmaceutical compositions can comprise various excipients, including without limitation stabilizers, antioxidants, colorants and diluents. In general, pharmaceutically acceptable carriers and additives are chosen such that side effects from the pharmaceutical compound are minimized and the performance of the compound is not compromised to such an extent that treatment is ineffective.

Compositions intended for oral use can be prepared according to any method known in the art for the manufacture of pharmaceutical compositions and such compositions can contain one or more agents selected from the group consisting of sweetening agents, flavouring agents, colouring agents and preserving agents in order to provide pharmaceutically elegant and palatable preparations. Tablets contain the active ingredient in admixture with non-toxic pharmaceutically acceptable excipients, which are suitable for the manufacture of tablets. These excipients may be, for example, inert diluents, such as calcium carbonate, sodium carbonate, lactose, calcium phosphate or sodium phosphate, granulating and disintegrating agents, for example, maize starch, or alginic acid, binding agents, for example starch, gelatin or acacia, and lubricating agents, for example magnesium stearate, stearic acid, or talc. Tablets can be uncoated or they can be coated by known techniques, for example to delay disintegration and absorption in the gastrointestinal tract and thereby provide sustained action over a longer period. For example, a time delay material such as glyceryl monostearate or glyceryl distearate can be employed. Formulations for oral use can also be presented as hard gelatin capsules wherein the active ingredients are mixed with an inert solid diluent, for example, calcium carbonate, calcium phosphate or kaolin, or as soft gelatin capsules wherein the active ingredients are present as such, or mixed with water or an oil medium, for example, peanut oil, liquid paraffin or olive oil.

Aqueous suspensions can be produced that contain the active materials in a mixture with excipients suitable for the manufacture of aqueous suspensions. Such excipients include suspending agents, for example, sodium carboxymethylcellulose, methylcellulose, hydroxypropylmethylcellulose, sodium alginate, polyvinylpyrrolidone, gum tragacanth and gum acacia; dispersing or wetting agents can be naturally-occurring phosphatides, for example lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyoxyethylene sorbitan monooleate. Aqueous suspensions can also contain one or more preservatives, for example, ethyl or N-propyl p-hydroxybenzoate, one or more colouring agents, one or more flavouring-agents, or one or more sweetening agents, such as sucrose or saccharin. Suitable aqueous vehicles include Ringer's solution and isotonic sodium chloride. Aqueous suspensions according to the invention may include suspending agents such as cellulose derivatives, sodium alginate, polyvinylpyrrolidone and gum tragacanth, and a wetting agent such as lecithin. Suitable preservatives for aqueous suspensions include ethyl and N-propyl p-hydroxybenzoate.

Oily suspensions may be formulated by suspending the active ingredients in an omega-3 fatty acid, a vegetable oil, for example, arachis oil, olive oil, sesame oil or coconut oil, or in a mineral oil such as liquid paraffin. The oily suspensions can contain a thickening agent, for example beeswax, hard paraffin or cetyl alcohol.

Sweetening agents, such as those set forth above, and flavouring agents can be added to provide a palatable oral preparation. These compositions can be preserved by addition of an antioxidant such as ascorbic acid.

Dispersible powders and granules suitable for preparation of an aqueous suspension by addition of water provide the active ingredient in admixture with a dispersing or wetting agent, a suspending agent and one or more preservatives. Suitable dispersing or wetting agents and suspending agents are exemplified by those already mentioned above. Additional excipients, for example sweetening, flavouring and colouring agents, can also be present.

Syrups and elixirs containing the compound of the invention can be formulated with sweetening agents, for example glycerol, sorbitol, or sucrose. Such formulations can also contain a demulcent, a preservative and flavouring and colouring agents.

Compositions of the present invention can optionally be supplemented with additional agents such as, for example, viscosity enhancers, preservatives, surfactants and penetration enhancers. Viscosity-building agents include, for example, polyvinyl alcohol, polyvinyl pyrrolidone, methylcellulose, hydroxypropykmethylcellulose, hydroxyethylcellulose, carboxymethylcellulose, hydroxypropylcellulose or other agents known to those skilled in the art. Such agents are typically employed at a level of about 0.01% to about 2% by weight of a pharmaceutical composition.

Preservatives are optionally employed to prevent microbial growth prior to or during use. Suitable preservatives include polyquaternium-1, benzalkonium chloride, thimerosal, chlorobutanol, methylparaben, propylparaben, phenylethyl alcohol, edetate disodium, sorbic acid, or other agents known to those skilled in the art. Typically, such preservatives are employed at a level of about 0.001% to about 1.0% by weight of a pharmaceutical composition.

Pharmaceutically acceptable excipients and carriers encompass all the foregoing and the like. The above considerations concerning effective formulations and administration procedures are well known in the art and are described in standard textbooks. See for example Remington: The Science and Practice of Pharmacy, 20th Edition (Lippincott, Williams and Wilkins), 2000; Lieberman et al., ed. , Pharmaceutical Dosage Forms, Marcel Decker, New York, N.Y. (1980) and Kibbe et al., ed. , Handbook of Pharmaceutical Excipients (7th Edition), American Pharmaceutical Association, Washington (1999).

Thus, in embodiments where the compound of the invention is formulated together with a pharmaceutically acceptable excipient, any suitable excipient may be used, including for example inert diluents, disintegrating agents, binding agents, lubricating agents, sweetening agents, flavouring agents, colouring agents and preservatives. Suitable inert diluents include sodium and calcium carbonate, sodium and calcium phosphate, and lactose, while cornstarch and alginic acid are suitable disintegrating agents. Binding agents may include starch and gelatin, while the lubricating agent, if present, will generally be magnesium stearate, stearic acid or talc. The pharmaceutical compositions may take any suitable form, and include for example tablets, elixirs, capsules, solutions, suspensions, powders, granules, nail lacquers, varnishes and veneers, skin patches and aerosols.

The pharmaceutical composition may take the form of a kit of parts, which kit may comprise the composition of the invention together with instructions for use and/or a plurality of different components in unit dosage form.

For oral administration the compound of the invention can be formulated into solid or liquid preparations such as capsules, pills, tablets, troches, lozenges, melts, powders, granules, solutions, suspensions, dispersions or emulsions (which solutions, suspensions dispersions or emulsions may be aqueous or non-aqueous). The solid unit dosage forms can be a capsule which can be of the ordinary hard- or soft-shelled gelatin type containing, for example, surfactants, lubricants, and inert fillers such as lactose, sucrose, calcium phosphate, and cornstarch.

Tablets for oral use may include the dispersion of the invention, either alone or together with pharmaceutically acceptable excipients, such as inert diluents, disintegrating agents, binding agents, lubricating agents, sweetening agents, flavouring agents, colouring agents and preservatives. Suitable inert diluents include sodium and calcium carbonate, sodium and calcium phosphate, and lactose, while corn starch and alginic acid are suitable disintegrating agents. Binding agents may include starch and gelatin, while the lubricating agent, if present, will generally be magnesium stearate, stearic acid or talc. If desired, the tablets may be coated with a material such as glyceryl monostearate or glyceryl distearate, to delay absorption in the gastrointestinal tract. Capsules for oral use include hard gelatin capsules in which the compound of the invention is mixed with a solid diluent, and soft gelatin capsules wherein the active ingredient is mixed with water or an oil such as peanut oil, liquid paraffin or olive oil.

The dispersions of the invention are tableted with conventional tablet bases such as lactose, sucrose, and cornstarch in combination with binders such as acacia, cornstarch, or gelatin, disintegrating agents intended to assist the break-up and dissolution of the tablet following administration such as potato starch, alginic add, corn starch, and guar gum, lubricants intended to improve the flow of tablet granulations and to prevent the adhesion of tablet material to the surfaces of the tablet dies and punches, for example, talc, stearic add, or magnesium, calcium, or zinc stearate, dyes, colouring agents, and flavouring agents intended to enhance the aesthetic qualities of the tablets and make them more acceptable to the patient.

Suitable excipients for use in oral liquid dosage forms include diluents such as water, milk and alcohols, for example, ethanol, benzyl alcohol, and the polyethylene alcohols, either with or without the addition of a pharmaceutically acceptably surfactant, suspending agent or emulsifying agent.

Posology

The preferred route of administration is oral administration. The dose of the composition for therapy or prophylaxis as described herein is determined in consideration of age, body weight, general health condition, diet, administration time, administration method, clearance rate, combination of drugs, the level of disease for which the patient is under treatment then, and other factors.

The desired dose is preferably presented as a single dose for daily administration. However, two, three, four, five or six or more sub-doses administered at appropriate intervals throughout the day may also be employed.

While the dose varies depending on the target disease, condition, subject of administration, administration method and the like, for oral administration as a therapeutic agent for the treatment of Duchenne muscular dystrophy in a patient suffering from such a disease is from 0.01 mg-10 g, preferably 10-400 mg, is preferably administered in a single dose or in 2 or 3 portions per day.

### Exemplification

The invention will now be described with reference to specific Examples. These are merely exemplary and for illustrative purposes only: they are not intended to be limiting in any way to the scope of the monopoly claimed or to the invention described. These examples constitute the best mode currently contemplated for practicing the invention.

### EXAMPLE 1

### Ezutromid Structure and General Properties

Ezutromid exhibits four polymorphic forms (Forms I-IV). The preferred form for use in the pharmaceutical compositions described herein is the amorphous form. Polymorph Form I is produced consistently by the manufacturing process described herein. It takes the form of a white to off-white crystalline solid with a melting point of 160-161° C.

Solubility of Ezutromid Form I Polymorph

The solubility of the drug substance at 20° C. in 18 different pharmaceutically-acceptable solvents has been assessed. In each case, about 25 mg of drug substance was allowed to equilibrate with 250 μL of solvent over 4 hours. The resulting saturated solutions were filtered and analysed by HPLC. The results are given in the Table below:

Solubility of Ezutromid Form I Polymorph

| | Solubility at | | Solubility at |
|---|---|---|---|
| Solvent | 20° C. (mg/mL) | Solvent | 20° C. (mg/mL) |
| 1-butanol | 0.35 | Toluene | 8.50 |
| 2-propanol | 0.47 | Heptane | 10.93 |
| 1-propanol | 0.79 | tert-butyl methyl ether | 11.67 |
| | | (TBME) | |
| 2-butanol | 0.85 | Dimethylsulfoxide | 13.20 |
| Methanol | 1.06 | Tetrahydrofuran | 25.92 |
| Ethanol | 1.14 | Acetone | 27.53 |
| Butan-2-one | 3.12 | 1,4-dioxane | 28.40 |
| Acetonitrile | 5.44 | Dimethylformamide | >33.81 |
| Ethyl acetate | 8.36 | Chloroform | >50.00 |

Additionally, ezutromid is practically insoluble in water (<1 μg/mL), and very slightly soluble in corn oil (0.6 mg/mL).

X-Ray Powder Diffraction

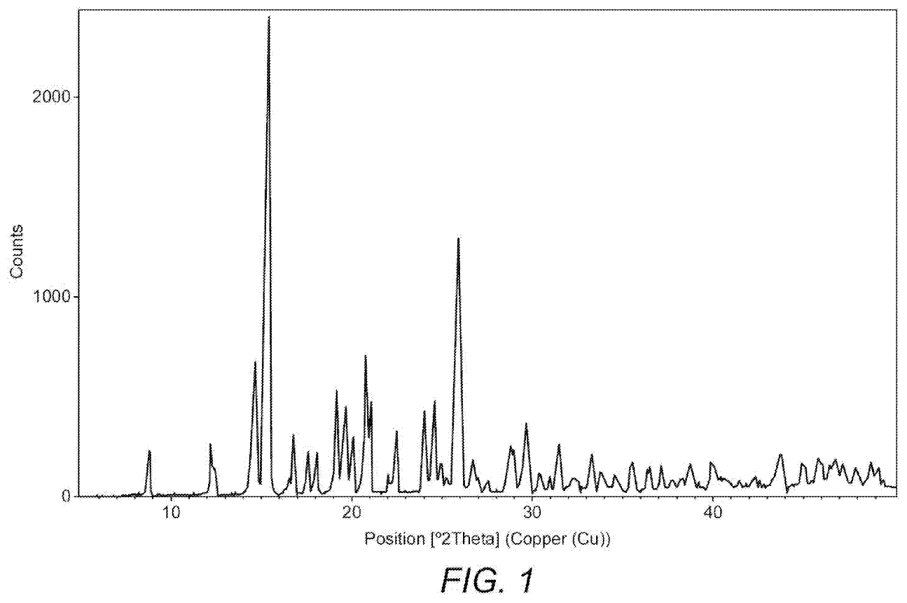

The XRPD pattern for Form I of the drug substance is shown in FIG. 1. The XRPD pattern shows a distinctive pattern of sharp peaks, demonstrating the crystalline nature of the solid.

Partition Coefficient

The water/octanol partition coefficient was determined with a ProfilerLDA isocratic chromatography system, using an octanol-coated column with octanol-saturated mobile phases. The results show that the drug substance is highly hydrophobic with logD=3.99 ±0.01 at pH 7.4.

Thermal Analysis

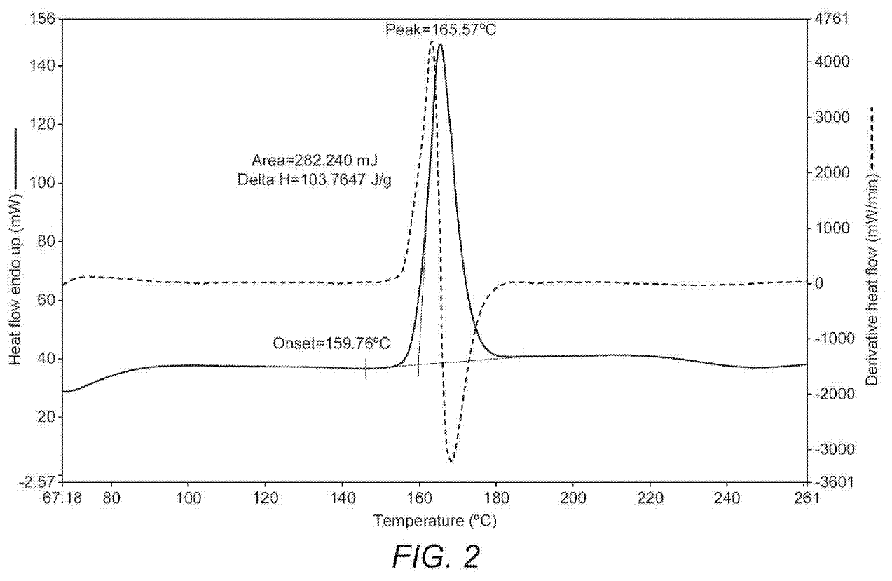

Differential scanning calorimetry (DSC) of the drug substance was performed using a Perkin-Elmer Diamond DSC unit. DSC was performed in a range from 0° C. to 200° C. under a helium purge to prevent oxidation, with a scan rate of 200° C. per minute. The DSC trace is given in FIG. 2. The results show a single melting event, with onset of melting at 159.8° C., and a latent heat of fusion of 103.8 J/g.

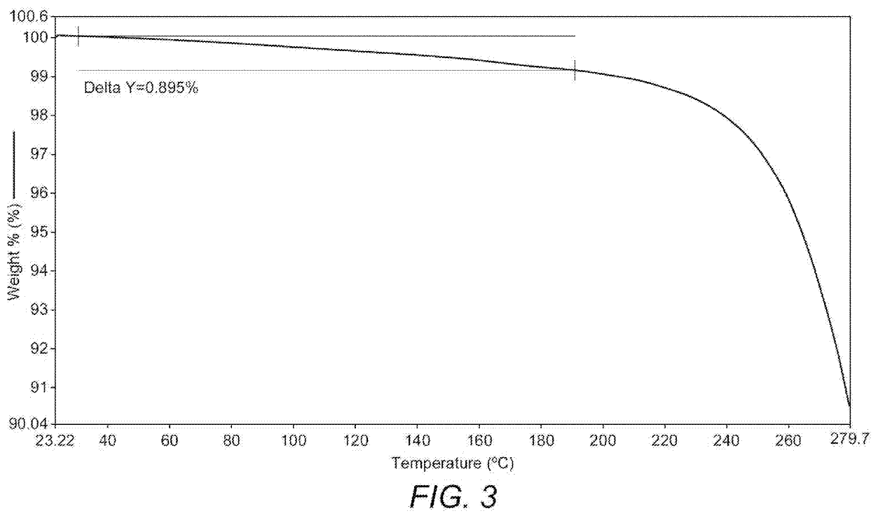

Thermal gravimetric analysis (TGA) of Form I shows a loss of about 0.9% of total mass when a sample is heated from 20° C. to 250° C. at a rate of 10° C./minute (see FIG. 3). A monohydrate would be expected to lose over 5.1% of its mass through loss of water, therefore this result indicates that Form I is an anhydrous, non-solvated form. The 0.9% mass loss is most likely due to residual moisture or solvent absorbed to the surface of crystals.

Additional Characterization Data

Form I was subjected to gravimetric vapour sorption analysis, ramping profile from 0 to 90% RH at 10% RH increments. The results demonstrate that the drug substance absorbs no more than 0.25% by weight of moisture up to 90% RH, and that this slight uptake is completely reversed under dry-air conditions. Based on these results, the drug substance is not hygroscopic.

### EXAMPLE 2

### Ezutromid Chemical Synthesis

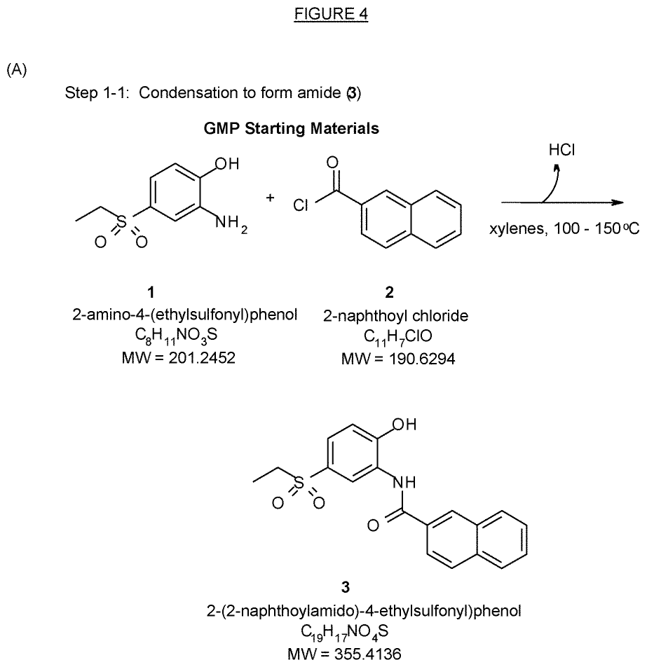

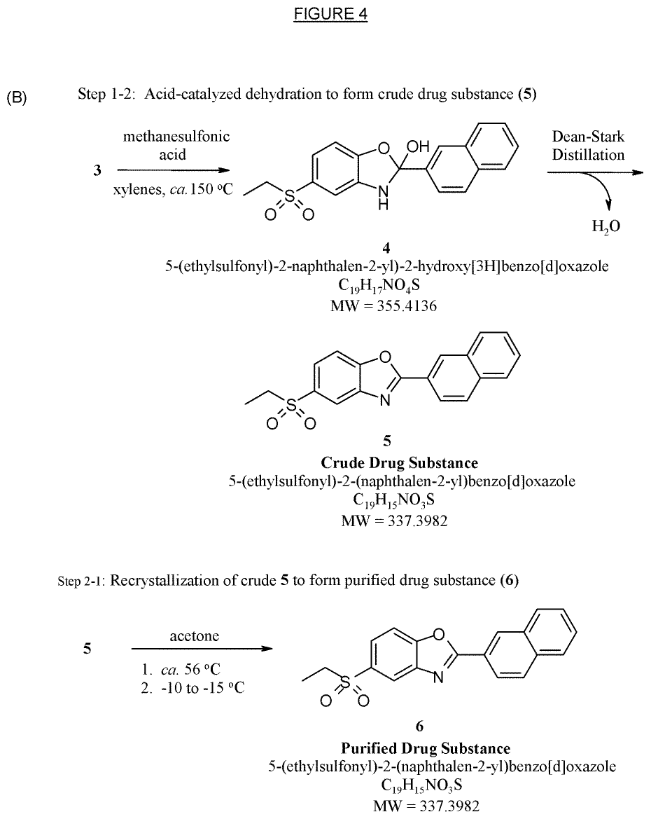

Ezutrornid is manufactured by chemical synthesis of the crystalline product, followed by jet-milling to adjust particle size. The chemical synthesis is depicted in FIG. 4. In brief, the crude drug substance is chemically synthesised via a two-step process. The crude drug substance is then purified, and sub-lots of purified drug substance are combined and subjected to jet-milling to reduce the particle size of the material and create the final drug substance lot.

Synthesis

In step 1 (1.8 kg scale), ezutromid is prepared via amide bond formation between the two GMP starting materials: 2-amino-4-(ethylsulfonyl)phenol (1) and 2-naphthoyl chloride (2) to give intermediate (3). This is followed by condensation performed in xylenes at 155° C., which leads first to cyclization (4), followed by dehydration to give a solution of the crude drug substance (5). Upon cooling, the product crystallises and is filtered and washed with Cert-butyl methyl ether (TBME) prior to vacuum drying.

In step 2 (1 kg scale), crude drug substance is purified by recrystallisation from acetone.

Each batch of purified drug substance is subjected to analysis to meet an intermediate specification (see Table below) prior to further processing. Purified drug substance sub-lots that meet release criteria are combined and subjected to jet-milling.

| | | Test | | |
|---|---|---|---|---|
| Process | Sample | parameter | Specification | Test method |
| Step 1-2 | Crude solid | Appearance | White to brown/ | Visual |
| (Crude drug | | | black solid | Assessment |
| substance) | | Identity | Conforms with | USP <761> |
| | | (¹H NMR) | reference | Ph.Eur. 2.2.46 |
| | | Purity | ≥70% | USP <621> |
| | | (HPLC) | (peak area) | Ph.Eur. 2.2.46 |
| Step 2-1 | Purified | Appearance | Off-white to | Visual |
| (Pre-milled | crystals | | tan solid | Assessment |
| drug | | Identity | Consistent with | USP <761> |
| substance) | | (¹H NMR) | reference | Ph.Eur. 2.2.33 |
| | | Identity | Consistent with | USP <197> |
| | | (FT—IR) | reference | Ph.Eur. 2.2.24 |
| | | Purity | ≥98% | USP <621> |
| | | (HPLC) | (peak area) | Ph.Eur. 2.2.46 |
| | | Residual | Xylenes | USP <467> |
| | | solvents | ≤500 ppm | Ph.Eur. 2.4.24 |
| | | | Acetone | |
| | | | ≤1000 ppm | |
| | | | TBME | |
| | | | ≤1000 ppm | |
| | | Heavy | ≤20 ppm | USP <231> |
| | | metals | | Ph.Eur. 2.4.8 |
| | | (as Pb) | | |
| | | Residue on | ≤1.0% | USP <281> |
| | | ignition | | Ph.Eur. 2.4.14 |
| | | (sulphated | | |
| | | ash) | | |
| | | XRPD | Form I | USP <941> |
| | | | | Ph.Eur. 2.9.33 |

Jet-Milling

One combined purified drug substance batch is subjected to particle size reduction by jet-milling to create one bulk drug substance batch.

Control of Materials: Specifications for GMP Starting Materials

The specifications for 2-amino-4-(ethylsulfonyl)phenol (1) and 2-naphthoyl chloride (2) are provided in the Tables, below. Where necessary, purification of 1 is achieved by hot filtration in acetone, followed by recrystallisation from propan-2-ol/TBME, and 2 is purified by distillation.

Specifications for 2-amino-4-(ethylsulfonyl)phenol

| Test parameter | Specification |
|---|---|
| Appearance | Tan-brown solid |
| Identity, ¹H NMR | Consistent with reference |
| Identity, FT—IR | Consistent with structure |
| Purity, HPLC | >98% (peak area) |
| Water content (Karl Fischer | <2.0% |
| titration) | |

Specifications for 2-naphthoyl chloride

| Test parameter | Specification |
|---|---|
| Appearance | White to yellow/green solid |
| Identity, ¹H NMR | Consistent with structure |
| Identity, FT—IR | Consistent with reference |
| Purity, HPLC | >98% (peak area) |
| Water content (Karl Fischer | <2.0% |
| titration) | |

Reagents, Solvents and Other Materials

Argon is accepted on the suppliers certificate of analysis. Xylenes, TBME, acetone and methanesuifonic add as reagent are passed on the suppliers certificate of analysis together with an identity test (FT-IR) and appearance against internal specifications.

Controls of Critical Steps and Intermediates

Prior to Step 2-1, recrystallisation from acetone, each batch of crude drug substance is tested to meet specified criteria. The process is also controlled at Step 2-2 (jet-milling). Before any pre-milled drug substance is combined to constitute a larger batch for jet-milling, each batch is tested to conform to in-process specifications. Any batch of purified drug substance that does not conform to standards or specifications may be reprocessed by resubmitting the batch to Step 2-1, recrystallisation from acetone, Final drug substance that has been jet-milled, but does not conform to standards or specifications, may also be reprocessed by subjecting the batch to Step 2-2.

In-process tests, limits and/or specifications are described in the Table below, Testing is performed in accordance with compendial methods (USP or Ph.Eur.).

In-Process tests performed during synthesis of the drug substance

| Process | Sample | Test parameter | Specification | Test method |
|---|---|---|---|---|
| Step 1-2 | Crude | Appearance | White to brown/ | Visual |
| (Crude | solid | | black solid | Assessment |
| drug | | Identity (¹H | Conforms with | USP <761> |
| substance) | | NMR) | reference | Ph.Eur. 2.2.33 |
| | | Purity (HPLC) | ≥70% (peak area) | USP <621> |
| | | | | Ph.Eur. 2.2.46 |
| Step 2-1 | Purified | Appearance | Off-white to tan | Visual |
| (Pre-milled | crystals | | solid | Assessment |
| drug | | Identity (¹H | Consistent with | USP <761> |
| substance) | | NMR) | reference | Ph.Eur. 2.2.33 |
| | | Identity | Consistent with | USP <197> |
| | | (FT—IR) | reference | Ph.Eur. 2.2.24 |
| | | Purity (HPLC) | ≥98% (peak area) | USP <621> |
| | | | | Ph.Eur. 2.2.46 |
| | | Residual | Xylenes ≤500 ppm | USP <467> |
| | | solvents | Acetone ≤1000 ppm | Ph.Eur. 2.4.24 |
| | | | TBME ≤1000 ppm | |
| | | Heavy metals | ≤20 ppm | USP <231> |
| | | (as Pb) | | Ph.Eur. 2.4.8 |
| | | Residue on | ≤1.0% | USP <281> |
| | | ignition | | Ph.Eur. 2.4.14 |
| | | (sulphated | | |
| | | ash) | | |
| | | XRPD | Form I | USP <941> |
| | | | | Ph.Eur. 2.9.33 |

Process Validation and/or Evaluation

The process described above has been performed under cGMP conditions for a total of 27 batches of pre-milled drug substance and two batches of final drug substance. The synthesis and purification steps demonstrate product consistency.

Crystalline Polymorphism and X-Ray Powder Diffraction

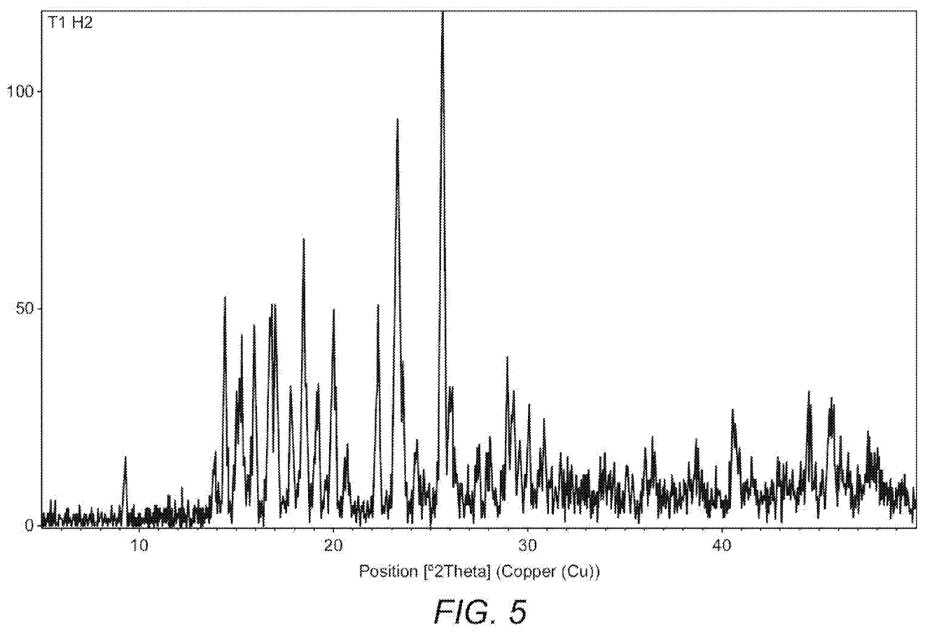

Two common crystalline polymorphs were identified by X-ray powder diffraction (XRPD) analysis during the development of ezutromid. These are identified as “Form I” and “Form II”. In addition, two other rarer forms, “Form III” and “Form IV”, have also been identified. Form I is the thermodynamically stable polymorph and is the form that results from recrystallisation in acetone, the procedure used in the manufacture of ezutromid as described above. Form II results from recrystallisation in xylene-IPA. The XRPD profiles of polymorphs Form I and Form II are displayed in FIGS. 1 and 5, respectively.

The identity of the polymorph in the drug substance is confirmed by XRPD analysis prior to use. Some differences in relative intensities between the observed profile and the reference spectrum of FIG. 1 may be observed: such differences are common with XRPD and may be due to variations in particle size, orientation of crystals in the instrument, and different instruments.

Infrared Spectroscopy

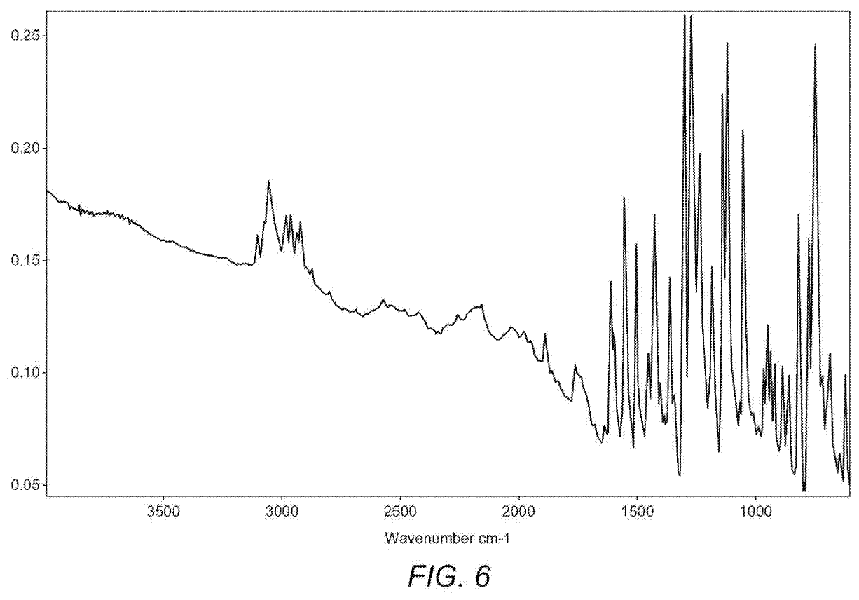

Fourier Transform Infrared (FT-IR) spectroscopy was performed using a Bruker Tensor 27 instrument fitted with a Miracle Pike ATR (Attenuated Total Reflectance) accessory. The FT-IR profile is shown in FIG. 6.

This spectrum is consistent with the expected structure of ezutromid. There are few peaks in the functional group region of the spectrum (wavenumbers≥1500 cm⁻¹). The peak at 3000 cm⁻¹ is likely to represent the aromatic C—H stretching vibration of the naphthalene and benzoxazole moieties. There is no evidence for hydroxyl groups in this region. Peaks near 1550 and 1600 cm⁻¹ may represent aromatic C═C bond stretching and C—stretching of the benzoxazole.

Raman Spectroscopy

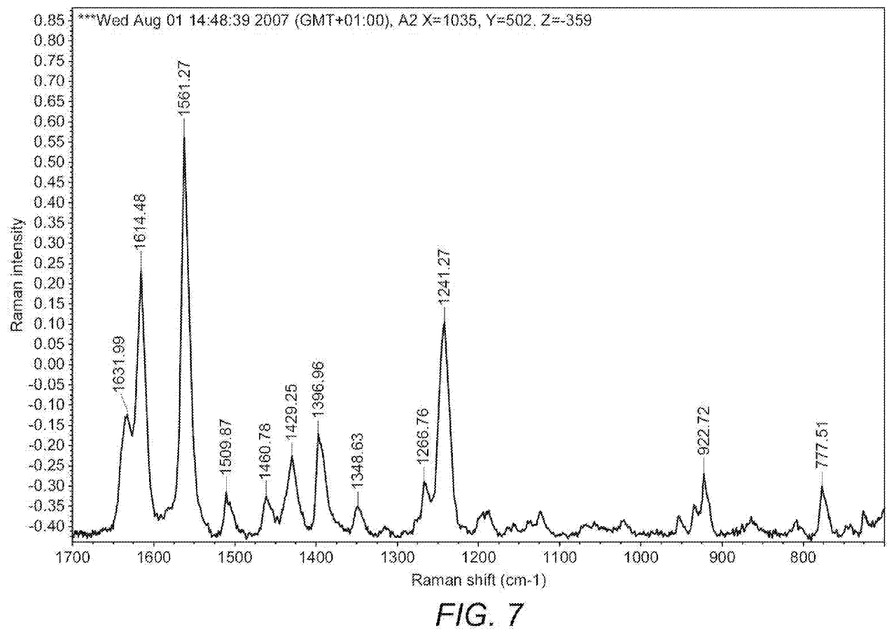

The Raman spectrum of ezutromid is shown in FIG. 7, and is consistent with the expected structure. The strong peaks between 1500 and 1650 cm⁻¹ are indicative of substituted aromatic ring structures. The peak at about 1400 cm⁻¹ suggests an aromatic ether (C—O—CH₂) stretch. Similarly, the peak near 1300 cm⁻¹ indicates the presence of an aromatic secondary amine.

Elemental Analysis

Elemental analysis of ezutromid drug substance for C, H and N was performed using a combustion method. Sulphur content was determined using ion-coupled plasma mass spectrometry (ICP-MS). The elemental analysis results agree with expected values calculated from the molecular formula of ezutromid (C₁₉H₁₅NO₃S), and thus provide evidence in support of the expected structure of the compound.

Elemental Analysis Results for Ezutromid

| | Expected value | Experimental value |
|---|---|---|
| Element | (% by mass)1 | (% by mass) |
| C | 67.64 | 67.28 |
| H | 4.48 | 4.23 |
| N | 4.15 | 4.20 |
| O | 14.23 | 14.812 |
| S | 9.50 | 9.48 |

¹Expected mass percentages were calculated from the molecular forrnula of ezutromid.
²Oxygen content was not determined experimentally. Oxygen percentages are calculated by subtraction of the values of the other elements from 100%.

### EXAMPLE 3

### Ezutromid ASD Formulation

Process Materials

| | | | Component |
|---|---|---|---|
| | | Component | Solution |
| Component | Component Purpose | Unit mg/g | Fraction (w/w) |
| SMT C1100 | Active Ingredient | 250 | 0.01 |
| HPMCAS | Excipient | 750 | 0.08 |
| | (Stabiliser/Solubility | | |
| | Enhancer) | | |
| Acetone | Process solvent | NA | 0.96 |
| Nitrogen | Process Gas (Drying) | NA | NA |

Process Description

A representative manufacturing process flow diagram is presented below. For clarity the process has been divided into three process sections: Feedstock solution preparation; Spray Drying Set-Up and Operation; Secondary Drying and Packaging.

Flowchart of the Medicinal Product Manufacturing Process and In-Process Controls

| | |
|---|---|
| Step | Description |

Feedstock solution preparation

| | |
|---|---|
| 1 | Dispense process solvent into process tank and turn on tank |
| | agitator |
| 2 | Dispense ezutromid drug substance and add to process tank |
| | and stir until visually clear |
| 3 | Dispense HPMCAS and add to process tank and stir until |
| | visually dissolved to generate the feedstock solution |

Spray drying set-up and operation

| | |
|---|---|
| 4 | Purge the start-up tank with nitrogen and dispense start-up |
| | solvent |
| 5 | Set-up the appropriate number of cyclone collection containers |
| 6 | Begin preheat of the spray dryer and prepare to switch to |
| | spray solution |
| 7 | Initiate spraying of the feedstock solution maintaining process |
| | parameters and begin collection of wet solid output |
| 8 | Remove required quantity of wet solid samples and transfer |
| | remaining bulk wet solids to a tray dryer |

Secondary drying and packaging

| | |
|---|---|
| 9 | Secondary dry the bulk wet solids until residual solvent is |
| | within specified limits |
| 10 | Bulk pack dried solids |

Feedstock Solution Preparation (Steps 1-3)

Acetone is added to the feedstock process tank and mixing is initiated. The solution temperature is maintained at 15-27° C. The specified batch quantity of ezutromid drug substance is added and the resulting suspension stirred until a clear solution results. The required quantity of HPMCAS is then added and mixed until dissolved.

Spray Drying Set-Up and Operation

The resulting feedstock solution is sprayed employing nitrogen as drying gas. Wet solids are collected in the cyclone collection containers that are replaced as and when needed. The spray-drying process is continued maintaining the process parameters and until the level of feedstock solution in the process tank is at foot-valve. The cyclone collector is then removed and replaced with the Spray Dry Tailings bottle the contents of which is recorded for weight and then disposed of. The required quantity of wet samples are collected according to the Sampling Plan and Product Record.

Secondary Drying and Packaging

The remaining bulk wet solids are collected from the cyclone collectors and transferred to a tray dryer for secondary drying at controlled temperature and humidity conditions. Secondary drying is continued for a predetermined amount of time at given conditions to ensure that the residual solvent levels are within specification.

### EXAMPLE 4

### Comparative Dissolution Profile

The Table below summarizes the dissolution profile of two formulations of ezutromid. Formulation A (invention) is an ASD prepared according to Example 3 (above).

Formulation B is a suspension of ezutromid having a particle size (D₅₀ particle size of 1.501 pm and a D₉₀ particle size of 3.368 μm).

| | Cₘₐₓ₉₀ | C₉₀ | AUC₉₀ |
|---|---|---|---|
| Group | (μg/mL) | (μg/mL) | (min* μg/mL) |
| A (invention) | 219 | 184 | 17200 |
| B | 28 | 24 | 2290 |

It can be seen that the dissolution profile of ezutromid after administration of ASD formulation A is better than that achieved after administration of the micronized formulation: both the AUC₉₀, Cₘₐₓ₉₀ and C₉₀ are significantly higher.

### EXAMPLE 5

### Comparison of Ezutromid Exposure (AUC and Cmax) Following Repeat Oral Administration of Ezutromid Formulations in Rats and Humans

The Table below summarizes the in vivo exposure (mean AUC and Cₘₐₓ) following repeat oral administration of ezutromid formulations in three distinct subject groups, including the target patient group (boys suffering from Duchenne muscular dystrophy).

Formulation A (invention) is an ASD prepared according to Example 3 (above).

Formulation B is a micronized aqueous suspension of ezutromid as described in WO/2013/167737, having a D₅₀ particle size of 1.501 μm and a D₉₀ particle size of 3.368 μm.

| | | Dose Normalized | Dose Normalized |
|---|---|---|---|
| Subject group | Formulation | Mean AUC(ng*h/mL) | Mean Cmax (ng/mL) |
| Wistar Rat | B | 26 | 5 |
| | A | 216 | 35 |
| Male Healthy | B | 134 | 17 |
| Volunteers | A | 1616 | 132 |
| DMD Boys | B | 448 | 30 |
| | A | 3510 | 281 |

It can be seen that ezutromid exposure in all three subject groups after administration of ASD formulation A is higher than that achieved after administration of the prior art micronized formulation B: both the mean AUC and Cₘₐₓ are much higher.

### EXAMPLE 6

### Improved Dose-Exposure Proportionality in DMD Patients

The Table below summarizes the in vivo exposure (weighted mean AUC) following repeat oral administration of ezutromid formulations in the target patient group (boys suffering from Duchenne muscular dystrophy).

Formulation A (invention) is an ASD prepared according to Example 3 (above).

Formulation B is a micronized aqueous suspension of ezutromid as described in WO12013/167737, having a D₅₀ particle size of 1.501 μm and a D₉₀ particle size of 3.368 μm.

| | | | Equivalent | Equivalent | Dosing |
|---|---|---|---|---|---|
| | Dose gr | AUC*** | Dose | Dose | Efficiency |
| Formulation | BID | ng/ml*hr | (Invention) | (Prior art) | Increase |
| B | 1.25 | 536 | 0.29 | — | 4.3 |
| B | 2.50 | 858 | 0.42 | — | 6.0 |
| A | 0.25 | 460 | — | 1.0 | 4.0 |
| A | 0.50 | 890 | — | 2.6 | 5.3 |
| A | 1.00 | 3347 | — | — | — |
| A | 2.00 | 5528 | — | — | — |
| A | 4.00 | 13146 | — | — | — |

***Weighted using Huber's criterion for outlier adjustment (see Analyst, December 1989, Vol 114 “Robust Statistics - How Not to Reject Outliers”, Analystical methods Committee, Royal Society of Chemistry)

The above data show that the mean exposure of a 1.25 gr dose in B is matched by a 0.29 gr dose in A, and the mean exposure of a 2.5 gr dose in B is matched by an 0.42 gr dose in A. Conversely, also as shown in the Table above, the requisite dose in B is 1 gr to match the comparable mean exposure delivered by a 0.25 gr dose in A. A 2.6 gr dose in B matches the exposure from a 0.5 gr dose in A.

Taken as a whole, these data indicate that doses of the formulation of the invention produce exposure levels that are proportional to the dose, whilst achieving a 5 fold greater exposure that doses delivered by the formulation of the prior art.

### EXAMPLE 7

### DMD Subject-Specific Exposure Limitations

Clinical studies using a micronized aqueous suspension of ezutromid as described in WO12013/167737 led to the discovery of a pronounced decrease in exposure of ezutromid in DMD boys compared to that observed in healthy volunteers. While the exact cause or causes for this are unknown at this time, administration of ezutromid in the form of micronized aqueous suspensions (as described in WO12013/167787) is associated with DMD subject-specific exposure limitations.

It has surprisingly been found that the formulation of the invention permits a dosing regimen with ezutromid that is exposure matched in both paediatric patients with DMD and in normal adult human volunteers.

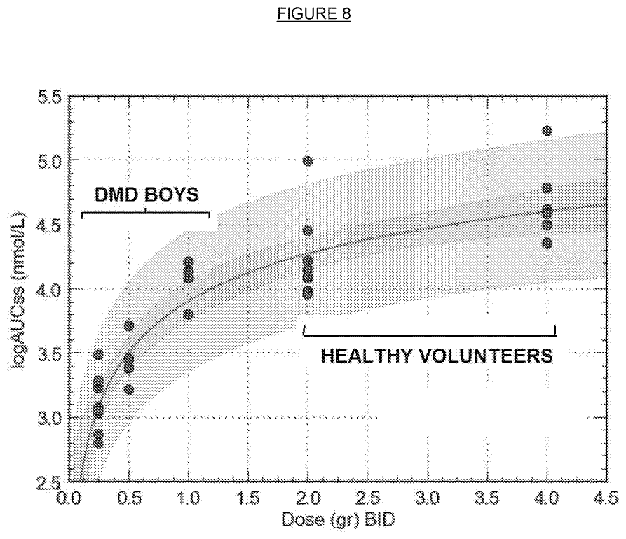

In particular, it was found that:
a) monotonic and continuous increases in exposure across the pediatric and adult populations over a five dose range from 0.25 to 4 gr were obtained, and
b) statistical predictability as the exposure in DMD boys falls in line with the findings in healthy adults when determined by fitting the dose-exposure relationship to the Hill equation (see FIG. 8).

These attributes are not reproducible with the corresponding data from dosing with the prior art formulation. As shown in FIG. 8, there is proportionality observed in exposures between DMD boys and healthy volunteers across a wide dose range.

This demonstrates that the formulation of the invention has mitigated or overcome mechanisms associated with the target patient population's disease condition that resulted in the sub-proportional ezutromid exposures observed with the micronized aqueous suspension formulation of the prior art.

### Equivalents

The foregoing description details presently preferred embodiments of the present invention. Numerous modifications and variations in practice thereof are expected to occur to those skilled in the art upon consideration of these descriptions. Those modifications and variations are intended to be encompassed within the claims appended hereto.

1. An amorphous solid dispersion comprising the compound 5-(ethylsulfonyl)-2-(naphthalen-2-yl)benzo[d]oxazole (ezutromid) and a polymer.

2. The dispersion of claim 1 wherein the polymer is in the form of a polymer matrix in which amorphous ezutromid is dispersed.

3. The dispersion of claim 1 wherein the polymer is a water soluble polymer.

4. The dispersion of claim 1 wherein the polymer is a solubilizing polymer.

5. The dispersion of claim 1 wherein the polymer inhibits amorphous ezutromid recrystallization in the solid-state and/or promotes supersaturation in the solution state upon dissolution.

6. The dispersion of claim 1 wherein the polymer comprises, or consists essentially of, a cellulosic or non-cellulosic polymer.

7. The dispersion of claim 6 wherein the polymer comprises, or consists essentially of, a cellulosic polymer, optionally selected from the group consisting of ionizable cellulosic polymers, non-ionizable cellulosic polymers, neutralized acidic cellulosic polymers and blends thereof.

8. The dispersion of claim 6 wherein the polymer comprises, or consists essentially of, a non-cellulosic polymer, optionally selected from the group consisting ionizable non-cellulosic polymers, non-ionizable non-cellulosic polymers, neutralized acidic non-cellulosic polymers and blends thereof.

9. The dispersion of claim 1 wherein the polymer is a chemically modified cellulose and/or cellulose ether.

10. The dispersion of claim 1 wherein the polymer is a chemically modified cellulose and/or cellulose ether selected from: alkylcellulose (for example methylcellulose, ethylcellulose and propylcellulose); hydroxalkylcellulose (for example hydroxymethylcellulose, hydroxyethylcellulose and hydroxypropylcellulose); hydroxyalkylalkylcellulose (for example hydroxyethylmethylcellulose (HEMC) and hydroxypropylmethylcellulose (HPMC)); carboxyalkylcellulose (for example carboxymethylcellulose (CMC), carboxymethylethylcellulose, carboxymethylhydroxyethylcellulose (CMHEC), hydroxyethylcarboxymethylcellulose (HECMC) and sodium carboxymethylcellulose); cellulose acetate phthalate (CAP); cellulose acetate trimellitate, hydroxypropylmethylcellulose acetate (HPMCA); hydroxypropylmethylcellulose phthalate (HPMCP); hydroxypropylmethylcellulose acetate succinate (HPMCAS), polyvinyl alcohols having repeat units in hydrolyzed form, polyvinyl pyrrolidone, poloxamers, polyvinylpyrrolidone, polyethylene glycol, polyethylene glycol based copolymer, polyacrylic acids and salts thereof, polyvinylalcohol, polyacrylamides copolymer, methacrylic acid copolymer, methacrylate copolymer, pectines, chitin and chitosan derivatives, polyphosphates, polyoxazoline, polysaccharides and mixtures thereof.

11. The dispersion of claim 1 wherein the polymer comprises, or consists essentially of, HPMCAS.

12. The dispersion of claim 11 wherein the HPMCAS is selected from subtypes L, M and H.

13-23. (canceled)

24. A pharmaceutical composition comprising the dispersion as defined in claim 1 and a pharmaceutically acceptable excipient.

25. A dosage form comprising the dispersion of claim 1, or pharmaceutical composition thereofof claim 21.

26-34. (canceled)

35. A process for producing a dispersion as defined in claim 1, pharmaceutical composition thereof or dosage form thereof comprising: (a) spray drying; (b) freeze drying; (c) hot melt extrusion, or (d) co-precipitation of said ezutromid and polymer.

36-40. (canceled)

41. A foodstuff comprising a dispersion as defined in claim 1, pharmaceutical composition thereof or dosage form thereof.

42. A composition produced, obtained, or obtainable by, the process of claim 35.

43. A dispersion as defined in claim 1, pharmaceutical composition thereof, dosage form thereof, foodstuff thereof or composition thereof for use in therapy or prophylaxis.

44. (canceled)

45. Use of a dispersion as defined in claim 1, pharmaceutical thereof, dosage form thereof, foodstuff thereof or composition thereof for the manufacture of a medicament for use in the treatment or prophylaxis of Duchenne muscular dystrophy or Becker muscular dystrophy.

46. A method for the treatment or prophylaxis of Duchenne muscular dystrophy or Becker muscular dystrophy in a patient in need thereof, comprising orally administering to the patient an effective amount of a dispersion as defined in claim 1, pharmaceutical composition thereof, dosage form thereof, foodstuff thereof or composition thereof.
